(19) United States
(12) Patent Application Publication
(10) Pub. No.: US 2025/0040500 A1
(43) Pub. Date: Feb. 6, 2025

(54) Suppression of Diseases and Abiotic Stress in Rice and Other Plants by Treatment with Avirulent Burkholderia glumae

(71) Applicant: Board of Supervisors of Louisiana State University and Agricultural and Mechanical College, Baton Rouge, LA (US)

(72) Inventor: Jong Hyun Ham, Baton Rouge, LA (US)

(73) Assignee: Board of Supervisors of Louisiana State University and Agricultural and Mechanical College, Baton Rouge, LA (US)

(21) Appl. No.: 18/787,255

(22) Filed: Jul. 29, 2024

Related U.S. Application Data
(60) Provisional application No. 63/530,287, filed on Aug. 2, 2023.

(51) Int. Cl. A01H 1/00; C12N 1/20; C12R 1/01

(52) U.S. Cl. CPC A01H 1/1255; A01H 1/1225; A01H 1/125; A01H 1/127; C12N 1/20; C12R 2001/01

(57) ABSTRACT

The avirulent strain A257 of the bacterial rice pathogen Burkholderia glumae is an effective priming material to reduce or suppress major diseases in rice and other plants. Protection includes that against bacterial panicle blight, sheath blight, and narrow brown leaf spot. A mutant derivative of A257, A257ΔqsmR, significantly reduces the risk that the priming agent might potentially revert to wild-type pathogenicity.

The benefit of the Aug. 2, 2023 filing date of U.S. provisional patent application Ser. No. 63/530,287 is claimed under 35 U.S.C. § 119 (e). The complete disclosure of the priority application is hereby incorporated by reference in its entirety.

This invention was made with government support under 2022-67013-36140 awarded by the National Institute of Food and Agriculture. The government has certain rights in the invention.

### REFERENCE TO AN ELECTRONIC SEQUENCE LISTING

The contents of the accompanying electronic sequence listing (File Name: HamA257.xml; Size: 6,307 bytes; Date of Creation: Jul. 16, 2024) is herein incorporated by reference in its entirety.

### TECHNICAL FIELD

This invention pertains to the suppression of diseases and abiotic stress in rice and other plants.

### BACKGROUND ART

Plants have multiple defense pathways to help protect against various biotic stresses (pathogens and pests) and abiotic stresses. ‘Priming’ is a plant defense mechanism, in which the prior exposure to certain stimuli leads to more rapid deployment of a plant's defenses following subsequent pathogen attacks or other challenges. “Defense priming” is associated with stimuli-induced systemic resistance (IR). The plant activates defenses such as systemic acquired resistance (SAR) or induced systemic resistance (ISR). Systemic acquired resistance is typically activated by chemical elicitors or pathogenic organisms. ISR is usually induced by beneficial microorganisms, such as plant growth-promoting rhizobacteria (PGPR). When a plant is triggered by a stimulus, it can enter a priming phase and then undergo various physiological changes, including changes in its gene expression, metabolism, and epigenetics—e.g., DNA and histone modification. Defense priming can be induced by beneficial microorganisms, usually those with plant growth promoting abilities such as PGPRs. The use of beneficial microorganisms can be environmentally-friendly, and cost-efficient, effective in enhancing the resistance and tolerance of plants against plant pathogens and abiotic stresses, particularly as compared to the application of synthetic compounds.

“Seed priming” can be used to stimulate seed germination and increase seed vigor. Seed priming techniques include hydropriming, osmopriming, redox priming, chemical priming, and hormonal priming. Seed priming techniques have been developed to enhance seed vigor and reduce environmental pressures on seedling growth and development. The effectiveness of a priming technique depends on factors including plant species, osmosis, the particular priming compound, the duration of priming, temperature, and seed conditions. Seed priming with hydration, also known as hydropriming, stimulates pre-germination metabolic processes and prepares the seed for radicle protrusion. Hydropriming aids in lowering the seed's physical barrier to imbibition, which in turn hastens seedling emergence. Hydropriming also helps with osmotic adjustment, metabolic repair after ingestion, and germination-enhancing metabolite synthesis.

Seed germination can be divided into three steps: The initial stage is seed imbibition. The second stage is the activation phase, including cell division; expression of nucleic acids, proteins, and ATP; the buildup of vital lipids; the formation of antioxidants; and the activation of DNA repair mechanisms. Water is rapidly absorbed by the seed during the third stage, which includes growth, cell elongation, and radicle protrusion. Seed priming is preferably applied at the first (seed imbibition) stage. The primed seeds are preferably redried before entering the second stage. Once seeds have entered the second state, the physiological processes of germination are effectively irreversible.

When plants grown from seeds primed with certain agents are later exposed to various biotic and abiotic stressors, more robust cellular defensive responses can result. These responses include the production of antimicrobial compounds, which help suppress pathogens, and the production of enzymatic antioxidants such as SOD, CAT, and POD, which help protect against reactive oxygen species (ROS) such as hydrogen peroxide, superoxides, and hydroxyl radicals that can be generated under stress conditions.

‘Defense priming’ and ‘seed priming’ can be combined into new strategies for augmenting the defense capacity of a crop. Seeds are treated with defense-inducing materials. For example, soaking seeds in a liquid bacterial medium can allow beneficial bacteria to penetrate the seeds. Biological control materials can enhance the overall growth and development of crops, as well as in increase their resistance to disease. Examples include the use of Trichoderma harzianum, Pseudomonas florescence, and Bacillus subtilis to reduce susceptibility to Fusarium spp., Rhizoctoniasolani, Pythium spp., or Sclerotium rolfsii. A study has shown the efficacy of seed bio-priming of chili seeds using Trichoderma asperellum against Fusarium solani and Pythium ultimum. Another study showed that cotton seeds bioprimed with fungal endophytes displayed enhanced seed germination and vigor, and increased resistance against Corynespora cassiicola and F. solani.

In ‘defense-priming,’ plants express enhanced activation of induced defense responses. A plant's defenses respond to biotic stresses via systemic acquired resistance (SAR), which can also be induced by exposure to avirulent or mild pathogens. For example, following a localized infection, the plant enters a primed phase. In the primed phase, upon subsequent challenge a faster, heightened, or prolonged defense response is activated. This much stronger defense response is attributed to “plant stress memory” or “defense priming”. During this phase, the plant undergoes various physical, transcriptional, metabolic, and epigenetic changes.

Seed priming is an emerging tool to help induce defense responses in a host plant. Plants that are seed-primed can exhibit cellular defense responses upon the exposure to various biotic and abiotic stimuli. Priming induces a plant's more rapid response and heightened resistance to future abiotic stresses or pathogen attacks. Seed priming can also result from prior exposure to beneficial microorganisms like rhizobacteria, mycorrhizal fungi and avirulent strains, or to natural compounds such as phytohormones and other non-toxic compounds.

One study has reported seed-biopriming effects of bacterial strains of Bacillus, Serratia, and Pseudomonas in reducing disease severity of rice blast from seed infection of Magnaporthe oryzae L. Another study employed Trichoderma, and observed upregulation of defense genes as well as of enzymes such as catalase, superoxide dismutase, and polyphenol oxidase, and the increased production of total disease-associated phenolics. See Amruta, N. et al. (2019). Bio-priming of rice seeds with novel bacterial strains, for management of seedborne Magnaporthe oryzae L. Plant Physiology Reports, 24 (4), 507-520; and Swain, H. et al. (2021). Seed Biopriming With Trichoderma Strains Isolated From Tree Bark Improves Plant Growth, Antioxidative Defense System in Rice and Enhance Straw Degradation Capacity [Original Research]. Frontiers in Microbiology 2021 Feb. 26; 12:633881.

Chin, J. M., et al. (2022). Biopriming chili seeds with Trichoderma asperellum: A study on biopolymer compatibility with seed and biocontrol agent for disease suppression. Biological Control, 165, 104819 reports a study in which the agent Trichoderma asperellum was bioprimed onto chili seeds, along with various biopolymers, to inhibit the growth of the pathogens Fusarium solani and Pythium ultimum.

El-Mohamedy, R. et al. (2013). Bio-priming seed treatment for biological control of soil borne fungi causing root rot of green bean (Phaseolus vulgaris L.). Intl. J. Agric. Technol, 9(3), 589-599 reports a greenhouse study in which the agents Trichoderma harzianum, Pseudomonas florescence, and Bacillus subtilis were primed onto green bean seeds to inhibit the growth of the pathogens Fusarium solani, Rhizoctonia solani, and Fusarium oxysporum.

Hussain, S., et al. (2016). Physiological and Biochemical Mechanisms of Seed Priming-Induced Chilling Tolerance in Rice Cultivars. Frontiers in Plant Science, 7, 116-116. https://doi.org/10.3389/fpls.2016.00116 examined the role of different seed priming techniques, such as hydropriming, osmopriming, redox priming, chemical priming, and hormonal priming, in enhancing the tolerance of rice to colder temperatures.

The bacterium Burkholderia glumae is a plant pathogen. B. glumae causes bacterial panicle blight, a major disease in rice. Karki H S, et al. (2012), Diversities in virulence, antifungal activity, pigmentation and DNA fingerprint among strains of Burkholderia glumae. PLOS One. 2012; 7 (9): e45376 identified and characterized a naturally avirulent strain of B. glumae called A257 (original strain name: 257sh-1). The whole genome of the A257 strain was reported in Lee, H. H., Lelis, T., Ontoy, J., Bruno, J., Ham, J. H., & Seo, Y. S. (2021). Complete Genome Sequence Data of Four Burkholderia glumae Strains Isolated from Rice Fields in the United States. Mol Plant Microbe Interact, 34 (11), 1324-1327.

Kim, J., et al. “Regulation of polar flagellum genes is mediated by quorum sensing and FIhDC in Burkholderia glumae.” Molecular microbiology 64.1 (2007): 165-179 describes the role of the transcriptional regulator QsmR in the pathogenicity of Burkholderia glumae.

It is challenging to predict in advance, before any experimental observations have been made, which candidate priming agents might be effective in defense priming or seed priming, against which pathogens, for which host plants.

### DISCLOSURE OF THE INVENTION

I have discovered that the avirulent strain A257 of the bacterial rice pathogen Burkholderia glumae is an effective priming material to reduce or suppress major rice diseases. Surprisingly, it is not only effective in enhancing protection against bacterial panicle blight (BPB), but also the rice diseases sheath blight (ShB) and narrow brown leaf spot (NBLS). In field trials in 2020 and 2021, A257 foliar spray treatment was effective to suppress BPB.

In 2021 and 2022, greenhouse and field trials showed that seed treatment with A257 significantly increased rice resistance to ShB, which is caused by a different pathogen, the fungus Rhizoctonia solani. Surprisingly, rice plants grown from A257-treated seeds also showed significantly reduced symptoms of naturally occurring NBLS, which is a serious rice disease caused by the fungal pathogen Cercospora janseana. A257 can be used as a biological agent for suppressing a broad range of both bacterial and fungal rice diseases through seed treatment, and against BPB through foliar spray.

I have also designed a mutant derivative of A257, A257ΔqsmR, which significantly reduces (almost to zero) the risk that the priming agent might potentially revert to wild-type pathogenicity. In greenhouse tests in 2023, the A257ΔqsmR mutant and the A257 strain showed comparable levels of efficacy in suppressing ShB. The A257ΔqsmR mutant provides the same beneficial priming effects, with almost zero potential risk of spontaneously-regained pathogenicity.

A257 can induce defense-priming in rice. Experiments have confirmed the activity of A257 both as a foliar spray and as a seed treatment to increase the resistance of rice plants to bacterial panicle blight (BPB), sheath blight (ShB), and narrow brown leaf spot (NBLS).

A whole genome DNA sequence analysis and genetics study revealed that the avirulence of the A257 strain resulted from a single amino acid substitution in the QsmR protein, a key regulatory factor required for pathogenesis by B. glumae. To hinder the potential reversion of A257 to virulence, I designed a new strain, A257ΔqsmR, which is a mutant derivative of A257 completely lacking the qsmR gene. Greenhouse tests have verified the effectiveness of A257ΔqsmR as a defense-priming agent in rice.

### BRIEF DESCRIPTION OF THE DRAWINGS

FIGS. 1(A) and 1(B) depict BPB disease severity for the rice varieties Bengal and CL111, respectively, following the application of A257 through foliar spraying, as compared to untreated plants.

FIGS. 2(A) and 2(B) depict yields for Bengal and CL111, respectively, following foliar treatment with A257, as compared to untreated plants.

FIGS. 3(A) and 3(B) depict BPB disease severity following treatment with A257 as a foliar spray alone, or as a combination of foliar application with seed treatment, respectively.

FIGS. 4(A) and 4(B) depict ShB disease severity in greenhouse and field trials, respectively, for A257-seed-treated plants and for untreated plants.

FIGS. 5(A) and 5(B) depict ShB disease severity in the sheaths and panicles, respectively, of A257-treated plants and for untreated plants.

FIG. 6 depicts NBLS disease severity in rice plants in field trials following A257 seed treatment, or no treatment.

FIG. 7 depicts ShB disease severity in greenhouse trials for rice plants grown from seeds treated with buffer, with A257, with A257ΔqsmR, or with a commercial seed-treatment fungicide.

FIGS. 8(A) and 8(B), depict BPB and ShB disease severity observations, respectively, following various treatments in greenhouse trials.

### MODES FOR CARRYING OUT THE INVENTION

In some embodiments, the strain A257 is used as a biological agent to suppress ShB and BPB disease severity in rice, without negative side-effects on overall plant growth and yield. Routes of application may be any of those known in the art, including for example foliar spraying or seed treatment. Results have shown that A257 treatment (by foliar spray or seed treatment) is effective to induce defense-priming in rice plants, so that they elicit more robust defense responses (e.g., expression of defense related genes and enzymes) upon pathogen attack.

For a BPB experiment conducted in 2020, A257 was applied as spray treatment to the rice varieties Bengal and CL111 one day before inoculation with a virulent strain of B. glumae. The rice plants that had been treated with A257 one day before inoculation showed significantly reduced disease symptoms as compared to untreated plants. Similar results were seen in 2021, when plants sprayed with A257 either one day before or one day after inoculation showed significantly lower disease rates as compared to untreated plants. In 2020 and 2021 field trials, the application of A257, whether as a foliar spray treatment alone or in combination with seed treatment, did not significantly affect yields. In 2020, Bengal plants that had been sprayed with A257 one day before inoculation with B. glumae showed a significantly higher yield as compared to untreated plants. Although the results were not statistically significant, CL111 plants pre-treated with A257 had higher yields compared to untreated plants in 2021 trials.

The efficacy of A257 as a seed treatment against ShB was evaluated in both greenhouse and field trials in 2021 and 2022. In these trials, rice plants grown from seeds treated with A257 showed significantly lower disease severity as compared to plants without seed treatment. In 2022, disease severity was scored separately for the sheath and panicle components. Even though the sheaths of the A257 seed-treated rice plants showed apparently lower disease scores, the scores were not statistically different as compared to the untreated plants. However, the panicles of the same plants showed a visually obvious and statistically significant difference; the A257 seed-treated plants exhibited lower panicle disease symptoms compared with the untreated plants.

Surprisingly, seed treatment with A257 also substantially reduced disease severity from natural NBLS infections during the 2021 growing season, when that fungal disease was prevalent in Louisiana rice fields. The experimental observations showed that A257 seed treatment can protect rice plants against a broad range of fungal pathogens responsible for major rice diseases throughout the growth cycle, from the early vegetative stage to the late reproductive stage of growth.

I hypothesize that defense-priming mediated by A257 will also be effective against one or more additional rice diseases (e.g., blast) and against insect damage (e.g., from rice water weevils and stemborers). I further hypothesize that A257 will also be effective to protect plants from abiotic stresses, such as drought. I further hypothesize that defense-priming mediated by A257 will also be effective in other crops, e.g., maize, wheat, and soybeans. Future greenhouse and field experiments, otherwise generally similar to those described herein, will be conducted to verify these hypotheses—Preliminary results from 2023 field trials with A257 seed treatments on soybeans appeared to show success in field trials against two fungal diseases, Cercospora leaf blight and Septoria brown spot (data not shown).

A preferred strain for use in this invention is A257ΔqsmR. This invention may also be practiced with other A257 variants in which the qsmR gene has been artificially deleted or otherwise artificially inactivated through methods known in the art, preferably with methods that minimize the likelihood of spontaneous reversion to a virulent genotype/phenotype. Other strains of the rice pathogen Burkholderia glumae may also be used in practicing this invention, wherein the qsmR gene has been artificially deleted or otherwise artificially inactivated through methods known in the art, preferably with methods that minimize the likelihood of spontaneous reversion to a virulent genotype/phenotype.

As used herein “artificially deleted or otherwise artificially inactivated” refers to a process, method, gene, nucleic acid, or strain in which direct or indirect human intervention has induced the deletion or inactivation of a gene or other nucleic acid sequence. The term “artificially deleted or otherwise artificially inactivated” is specifically intended to exclude products of nature. As two examples: (1) The strain A257 was originally discovered occurring naturally in a rice field. The unmodified strain A257 would not itself be considered to contain a gene that had been “artificially deleted or otherwise artificially inactivated.” (2) By contrast, the strain A257ΔqsmR was intentionally designed and artificially prepared in the laboratory using molecular techniques to remove the qsmR gene from the genome. The resulting A257ΔqsmR strain is therefore considered to be a strain in which the qsmR gene has been “artificially deleted or otherwise artificially inactivated.”

### Materials and Methods

### Rice Cultivars

Two rice (Oryza sativa) varieties, CL111 and Bengal, were used in various experiments in 2020, 2021, and 2022. Both of these varieties are susceptible both to bacterial panicle blight (BPB) and to sheath blight (ShB). CL111 is a very early, short-stature, long-grain, herbicide-tolerant (Clearfield®) variety. Bengal is an early-maturing, semidwarf, medium-grain, conventional (non-herbicide-tolerant) variety. The rice seeds were obtained from the LSU AgCenter H. Rouse Caffey Rice Research Station (HRCRRS), where both of these varieties had initially been developed.

### Bacterial Strains and Culture Conditions

A257 and A257ΔqsmR cultures were stored at −80° C. until used. After thawing, the strains were first grown on LB agar at 37° C. The bacterial cells were then inoculated in LB broth or LB agar for preparation of a cell suspension.

Foliar Spraying with A257

In 2020 and 2021 field studies, A257 was tested for its efficacy in suppressing BPB by foliar spraying on rice panicles, prior to pathogen inoculation. The A257 strain was grown overnight on LB agar plates at 37° C. A bacterial suspension at a concentration of OD₆₀₀=0.2 (˜1.0×10⁸ CFU/mL) was prepared by collecting the cell culture from LB agar plates and suspending it in 1 L of 10 mM MgCl₂ buffer. Then Tween 20 was added to the inoculum suspension at a 0.2% concentration (v/v). The suspension was then applied to Bengal and CL111 rice plants one day before or after the plants were inoculated with the pathogenic strain Burkholderia glumae 336 gr-1.

Bio-Priming of Rice Seeds with A257

In 2021 and 2022, A257 was evaluated in field trials with rice variety CL111. Its disease-suppressing (defense-priming) efficacy against BPB and ShB was assessed.

Prior to treatment with A257, the CL111 seeds were surface-sterilized by: soaking in 95% ethanol for 3-5 min, rinsing with sterile distilled water 5 times, soaking in 3% hydrogen peroxide for 30 sec, and a final rinsing with sterile distilled water 5 times. The surface-sterilized seeds were then air-dried for 30 min. For the seed treatment preparation, the A257 strain was initially grown on a freshly prepared LB agar plate for 24 h. Then a loopful of the grown bacteria was transferred to sterilized LB broth and incubated overnight in a shaking incubator at 37° C. at 190 rpm. Following incubation, the culture was resuspended in 10 mM MgCl₂ buffer, adjusting the final concentration to OD₆₀₀=2 (˜10⁹ CFU/ml). The surface-sterilized seeds were soaked in the bacterial suspension for 1 hour. The soaked seeds were air-dried for 1-4 days. The seed drying step helps prevent pre-germination. The treated seeds are ready to be planted once they are dried. The same procedure, using the 10 mM MgCl₂ buffer alone, was used as a negative control. The same procedure was used for both the A257 and A257ΔqsmR strains.

The elapsed interval between seed treatment and planting depended on the weather. At times when it was impractical to plant seeds immediately following overnight drying, the seeds were kept on a laboratory bench for 4 days to dry and inhibit possible decay. In the 2021 field study, all treated rice seeds were planted in the field 32 days after a 4-day drying period.

In 2022, the same seed treatments were again used with CL111 rice seeds. The A257-treated and -untreated seeds (control) were planted in the field 8 days after a 4-day dying period. Greenhouse tests were conducted with the seeds after overnight drying.

### Inoculum Preparation and Inoculation

A pure culture of overnight-grown B. glumae 336 gr-1 (a virulent strain) was used to inoculate CL111 and Bengal rice plants in the field in 2020. Two inoculation methods were used. For the CL111 variety, a pathogen inoculum suspension at a concentration of OD₆₀₀=0.1 (˜0.5×10⁸ CFU/ml) was sprayed onto plants at the 30% heading stage. Each plot (1.2 m×4.3 m) was sprayed with approximately 500 mL of the B. glumae suspension. The same concentration of the inoculum was applied to rice plants of Bengal variety during the booting stage using a metal brush dipped in the inoculum suspension. These plants were wounded from the stem through the tip of the leaves. Uninoculated plants of CL111 and Bengal varieties were used as controls.

For sheath blight testing, an inoculum of the fungal pathogen Rhizoctonia solani was prepared according to the method of Shrestha, B. K., Karki et al. (2016). Biological Control Activities of Rice-Associated Bacillus sp Strains against Sheath Blight and Bacterial Panicle Blight of Rice. PLOS One, 11 (1). Briefly, a pure culture of R. solani was initially grown on potato dextrose agar (PDA) plates for 7 days at 30° C. The PDA culture was then added to a rice hull medium. To prepare the rice hull medium, 1.5 L of a 1:2 (v/v) rice grain:hull mixture was saturated with tap water in a 2 L flask, and the mixture was autoclaved at 15 psi for 30 min at 121° C. The sterilized rice hull medium in a 2 L-flask was then mixed with a ˜16 cm² PDA plug containing 7-day old R. solani mycelia, followed by incubation at 25° C. for 10 days. After ten days, 1×-2× volume of a new sterilized rice grain:hull (1:2 v/v) mixture was added to the culture, and the culture was then spread uniformly over a clean brown paper and covered with a clean plastic sheet at room temperature. This culture was then used for field inoculation after 24 h incubation. The inoculum of R. solani was applied by hand, sprinkling it near the base of the rice plants at the tillering stage of growth. For greenhouse tests, PDA plugs ˜1 cm² from a 7-day old R. solani culture were attached to the rice sheath, and wrapped with a piece of aluminum foil. Then the inoculated plants were kept in a humid chamber in a greenhouse for 48 hours.

### Field Experiments

The defense-priming efficacy of A257 against ShB and BPB was evaluated as a foliar spray or a seed treatment for three consecutive years (2020-2022) in field trials at the H. Rouse Caffey Rice Research Station (Rayne, LA). The field trials were arranged in a randomized complete block design with 4 replications/blocks per treatment. Each plot measured 1.2 m×4.3 m. 70 grams of seeds were drill-seeded in each plot. The design of these trials is summarized in Table 1.

TABLE 1
Overview of Field Trials 2020-2022.

| | | Application |
|---|---|---|
| | | (Days after |
| Year | Activity | sowing (DAS)) |
| 2020 | Fertilization (Pre-flooding) | 49 DAS |
| | Herbicide applications | 21, 28, 48, |
| | | and 85 DAS |
| | Insecticide | Seed-treatment |
| 2021 | Fertilization (Pre-flooding) | 52 DAS |
| | Herbicide applications | 23 and 52 DAS |
| | Insecticide | 1 DAS |
| 2022 | Fertilization (Pre-flooding) | 37 DAS |
| | Herbicide applications | 2 and 24 DAS |
| | Insecticide | Seed-treatment |

### Disease Severity Assessment

For BPB, disease severity was scored for the same two cultivars from one to three weeks after pathogen inoculation, using the 0-9 scale of Shahjahan, A. et al. (2000). Panicle blight. Rice J, 15, 26-29; in which 0=no symptoms, 1=spikelet discoloration limited to ¼ of the panicle, 3=spikelet discoloration limited to ½ of the panicle, 5=spikelet discoloration more than ½ of the panicle, 7=spikelet discoloration 75% of the panicle, and 9=spikelet discoloration more than 90% of the panicle, erected and unfilled panicle. Disease severity data were collected every week following inoculation. Thus in each plot disease scores were recorded four times. The disease severity score was calculated as the mean score per panicle.

For assessing ShB disease severity, symptoms were evaluated beginning one to three weeks after inoculation and continuing until maturity, using the 0-9 scale in which 0=no disease symptoms, 1=<1% sheath area affected, 3=1-5% sheath area affected, 5=6-25% sheath area affected, 7=26-50% sheath area affected, and 9=51-100% sheath area affected, per IRRI (2002). Standard Evaluation System for Rice. International Rice Research Institute, Manila, Philippines. Disease severity data were collected every week. Thus four disease scores were recorded for each plot. The disease severity score was calculated as the mean score per plant.

Disease severity data for greenhouse experiments were gathered using the same disease scoring systems.

### Statistical Design and Analysis

The field experiments were arranged in a randomized complete block design, with 4 replications/blocks per treatment. Greenhouse experiments were arranged in a complete randomized design, with three replications per treatment.

Comparisons of mean disease severity and yield data were analyzed either by T-test or Analysis of Variance (ANOVA). Post-hoc testing was done using Tukey's HSD at a significance level of 0.05. All data were statistically analyzed in R, and the figures were generated with ggplot2.

Deletion of the qsmR Gene from the Strain A257 to Generate the Mutant Strain A257ΔqsmR

To minimize the risk of reversion to a wild-type, virulent phenotype, I designed a variant of the A257 strain in which the qsmR gene had been deleted. The qsmR deletion mutant A257ΔqsmR was generated through double-crossover homologous recombination in the flanking regions of the qsmR gene. The genomes of three virulent strains and of the 257-sh1 (=A257) nonpathogenic strain of B. glumae are reported in Lee, H. H et al. (2021). Complete Genome Sequence Data of Four Burkholderia glumae Strains Isolated from Rice Fields in the United States. Mol Plant Microbe Interact, 34 (11), 1324-1327.

A DNA construct used for deletion of qsmR, pKKSacBΔqsmR, was prepared. A 458-bp upstream region and a 469-bp downstream region were amplified with the primer sets qsmrBamHIUL/qsmrSpeIUR and qsmrSpeIDL/qsmrXbaIIDR1 (Table 2). The resulting upstream and downstream PCR products were then cloned into the plasmid pSC-A-amp/kan to generate pSCqsmRU and pSCqsmRD, respectively. The upstream region in pSCqsmRU was further cloned into pKKSacB using the BamHI and SpeI restriction sites to generate pKKqsmRU. The qsmR downstream region in pSCqsmRD was cloned into pKKqsmRU using the SpeI and Xba/I restriction sites to obtain pKKSacBΔqsmR. This DNA construct was transformed into E. coli S17-1λpir through electroporation, and then introduced into B. glumae A257 via triparental mating, using the helper strain E. coli HB101 (pRK2013:Tn7). After this transformation step to introduce pKKSacBqsmRD into A257 cells, the qsmR deletion mutant derivative of A257, A257ΔqsmR, was obtained through selection using kanamycin resistance and sucrose sensitivity marker genes. The complete deletion of the qsmR gene was confirmed by PCR using the primer set qsmrDLcheckF and qsmrDLcheckR (Table 2).

Essentially the same procedures may also be employed to generate qsmR deletion mutants of other B. glumae strains, which then may also be used in practicing this invention.

TABLE 2
DNA primers used for the DNA construct
(pKKSacBΔqsmR) to generate a qsmR deletion mutant
of A257.

| Name | Sequence (5′ - 3′) | SEQ ID NO |
|---|---|---|
| qsmrBamHIUL | GGATCCGGGATCCGTCGATTT | SEQ ID NO 1 |
| | CATCGCCAATTT | |
| qsmrSpeIUR | ACTAGTGGGACTAGTCGGTCG | SEQ ID NO 2 |
| | CTGCTTTATTCAGT | |
| qsmrSpeIDL | ACTAGTGGGACTAGTGAATGG | SEQ ID NO 3 |
| | CTGCTCGAGACT | |
| qsmrXbaIIDR1 | TCTAGAGGGTCTAGATCATGT | SEQ ID NO 4 |
| | TCGATCTGGCTGAC | |
| qsmrDLcheckF | CGTGCTAGAACCTGAGAGAC | SEQ ID NO 5 |
| qsmrDLcheckR | ATCGTCCAGAGCACTTTCT | SEQ ID NO 6 |

### Results

### Effects of A257 Treatment on BPB

### 2020 Data on BPB (Effects of A257 Foliar Spray)

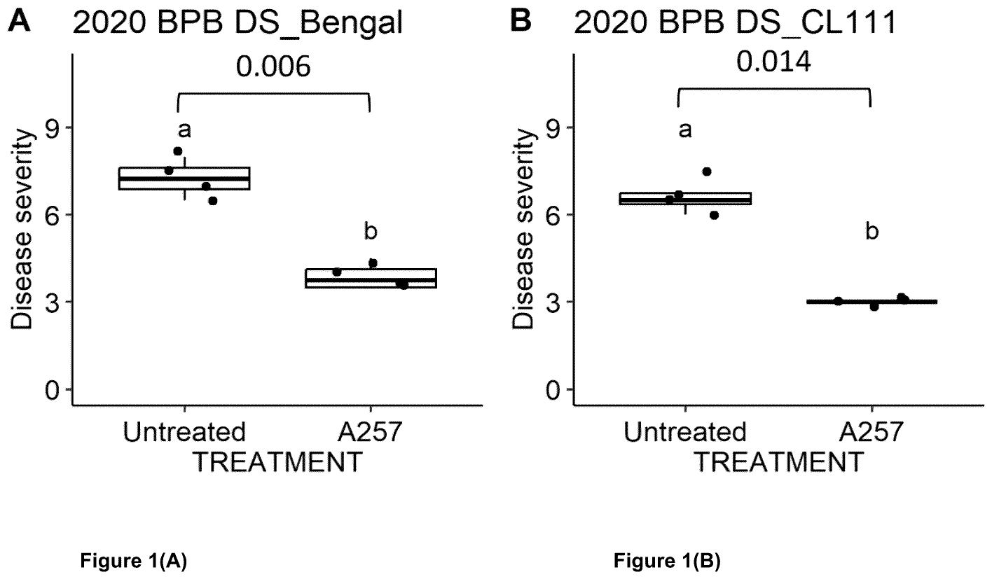

The symptoms of bacterial panicle blight disease, such as panicle discoloration and unfilled grains, are typically observed around 21 days post-inoculation (dpi). The application of A257 by foliar spraying, one day before pathogen inoculation, resulted in significantly lower disease severity for treated plants as compared to untreated plants, for both the Bengal and CL111 varieties. See FIGS. 1(A) and 1(B). The comparison of mean disease severity was analyzed using the T-test.

Plants that had been treated with A257 by foliar spraying showed clean panicles, similar in appearance to those of uninoculated rice plants. By contrast, untreated plants inoculated with the pathogen exhibited BPB symptoms such as discolored grains, erected and mostly unfilled panicles.

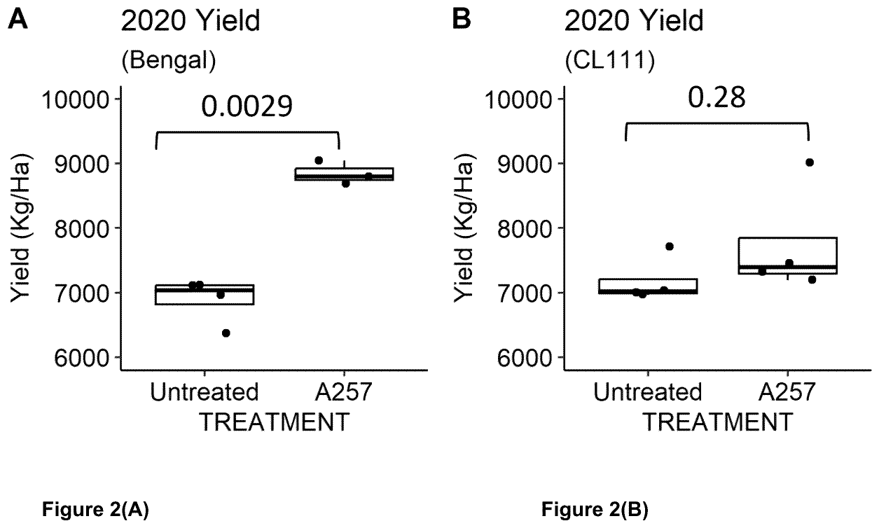

In these trials, the foliar application of A257 appeared to provide greater benefit to Bengal than to CL111. The Bengal variety treated with A257 showed significantly higher yield (9182 kilograms per hectare, kg/ha) compared to that for untreated (control) plants (6890 kg/ha). See FIG. 2(A). The CL111 variety treated with A257 also showed a higher yield (7745 kg/ha) than the control (7178 kg/ha); however, for CL111 the difference was not statistically significant. See FIG. 2(B).

FIGS. 2(A) and 2(B) depict yield data (kg/ha) for Bengal and CL111, respectively, following foliar treatment with A257 as compared to untreated (control) plants. Comparisons of means were analyzed using the T-test. Each treatment had four replications; each replication was a plot 1.2 m×4.3 m.

### 2021 Data on BPB (Effects of Foliar Spraying, and Effects of Foliar Spraying Plus Seed Treatment)

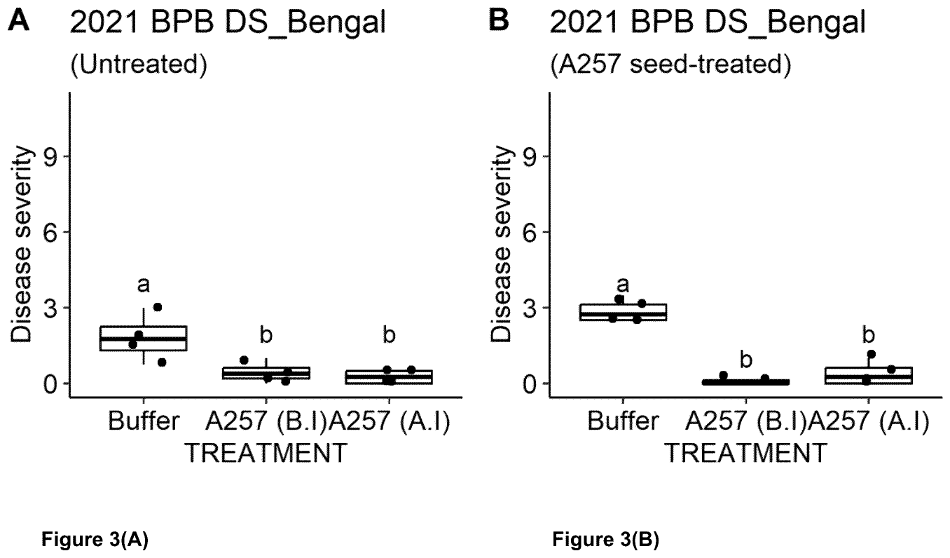

In 2021, the effects of A257 on BPB disease under field conditions were investigated following foliar-only spraying, or following a combination of foliar spraying plus seed treatment. In 2021, disease pressure from BPB in the field was low overall. Applying A257 as a foliar spray alone (FIG. 3(A)) or as a combination of foliar application plus seed treatment (FIG. 3(B)) was effective to suppress BPB. Disease suppression was similar with foliar spray one day before (B.I) or one day after (A.I) inoculation. Buffer treatment (one day before inoculation) was used as control. Disease severity was analyzed using ANOVA. Different letters in the Figures indicate significant differences according to the post-hoc test Tukey's HSD at α=0.05. In these trials we did not observe a significant effect of A257 on BPB when the A257 was applied through seed treatment only. Yield data showed no significant differences among the treatments, presumably due to the low overall disease pressure in 2021 (yield data not shown).

### Effects of A257 Treatment on ShB

The effects of A257 seed treatment in suppressing sheath blight were evaluated in 2021 and 2022, under both greenhouse and field conditions. As with the bacterial panicle blight experiments, these experiments were conducted in the greenhouses and rice fields of the H. Rouse Caffey Rice Research Station (Rayne, Louisiana). The CL111 rice variety was used for the field trials, and the Bengal variety was used for the greenhouse experiments.

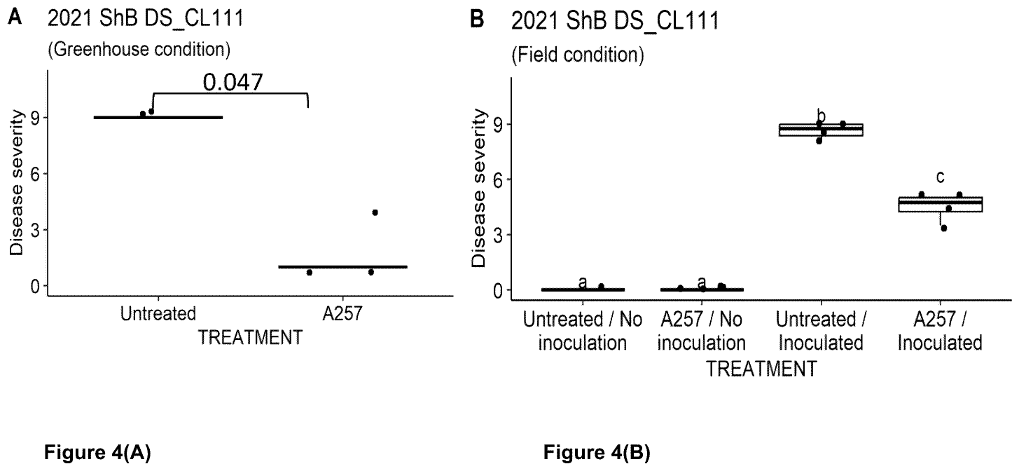

In the greenhouse trials, the seed-treated plants exhibited significantly lower ShB disease severity as compared to the untreated control plants (FIG. 4(A)). The untreated plants looked dried and almost dead following ShB pathogen-inoculation, and they produced few and mostly unfilled tillers. By contrast, the A257 seed-treated rice plants looked healthier, and produced more tillers, and more filled tillers, as compared to the untreated plants.

Similar to the findings of the greenhouse trial, the A257 seed-treated plants in the field also showed significantly lower disease severity as compared to untreated plants 5 weeks after inoculation (FIG. 4(B)). Thus, in both greenhouse and field conditions, A257 seed treatment showed significant ShB-suppressing effects. However, despite the disease-suppression effects of A257, we did not find significant effects on yield, due to the wide range of variation among replicates (data not shown).

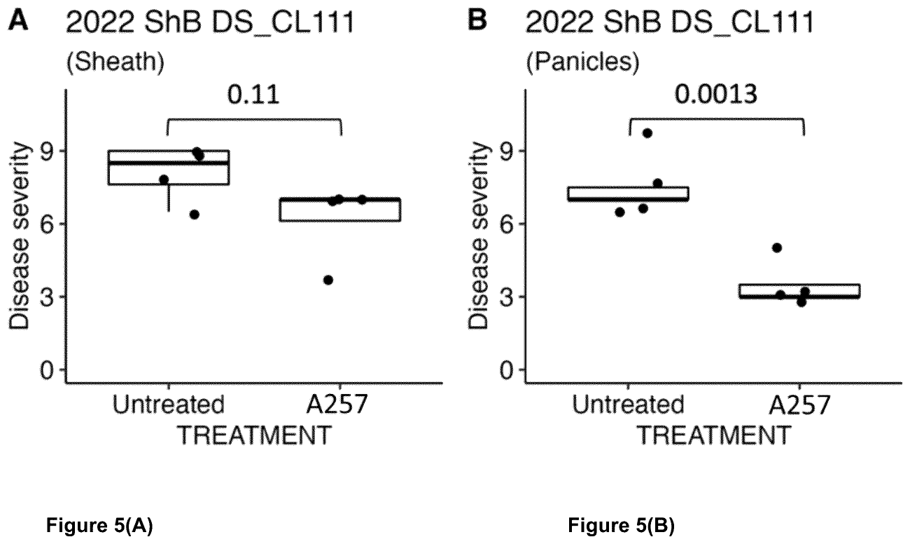

In 2022, a field trial was conducted at the H. Rouse Caffey Rice Research Station to evaluate the efficacy of A257 seed treatment against ShB disease. We observed that the A257 seed-treated plants exhibited lower disease symptoms. However, the differences in disease severity on rice sheaths was not statistically significant. (FIG. 5(A)). The mean comparison of disease severity was analyzed using T-test. Surprisingly, though, a significant difference was seen in ShB symptoms in panicles at a later growth stage, comparing plants grown from untreated seeds and those grown from A257-treated seeds (FIG. 5(B)).

Unfortunately, we did not obtain reliable yield data for the 2022 field trials, due to an error in plot identification while operating the harvesting machine.

### Effect of A257 Seed Treatment on Narrow Brown Leaf Spot (NBLS) Disease

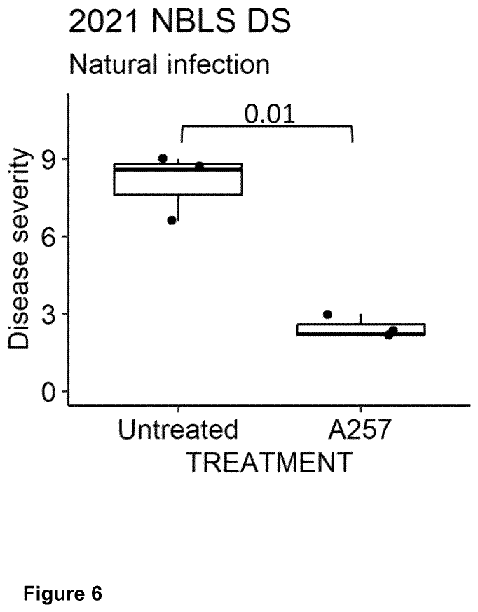

In 2021 there was an unexpectedly severe outbreak of NBLS at the H. Rouse Caffey Rice Research Station test plots. NBLS is a chronic rice disease worldwide, and is becoming more serious in the southeastern U.S. NBLS is caused by the fungal pathogen Cercospora janseana. Surprisingly, the A257-treated CL111 rice plants suffered much less damage from the 2021 NBLS epidemic as compared to untreated plants. FIG. 6 shows the effect of A257 seed treatment on natural infection by NBLS under field conditions in 2021. A substantial and significant reduction in symptoms from NBLS was seen in the rice plants from A257-treated seeds. These observations indicate that seed treatment with A257 can protect rice plants from a broad range of fungal diseases, not just from ShB—a surprising and unexpected result.

Generation of a Mutant Derivative of A257 Lacking the qsmR Gene

Genetic analysis of A257 revealed a variation in the qsmR gene thought to be of particular significance. The qsmR gene is a key regulatory gene in B. glumae that is essential for pathogenesis. A single point mutation in the qsmR gene is believed to be responsible for the avirulence phenotype in the A257 strain, a mutation resulting in the substitution of threonine with lysine at position 50. (See priority application Ser. No. 63/530,287, FIG. 10 and the accompanying text.) However, the phenotype reverted to strong virulence if a copy of the qsmR gene from the virulent strain 336 gr-1 was introduced into A257. These observations suggest that simply applying the unmodified A257 strain to rice seeds runs the risk of possible spontaneous reversion to wild-type virulence, with only a single nucleotide shift. To substantially reduce the likelihood of such spontaneous reversion, I designed a mutant derivative of A257 lacking the entire qsmR coding region. The qsmR gene was deleted by inducing a double homologous recombination with a purpose-made DNA construct, to produce the mutant A257ΔqsmR. This mutant of A257 has a precise deletion of the qsmR gene.

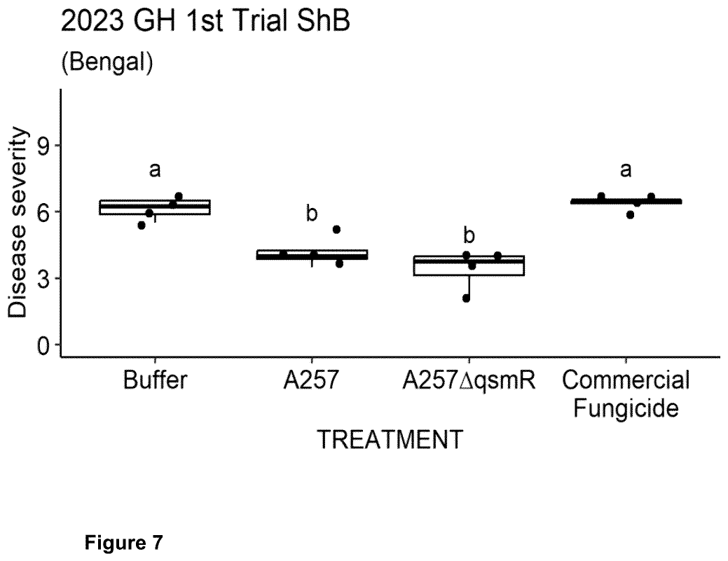

Preliminary seed treatment experiments in the greenhouse showed that A257ΔqsmR induced a similar protective effect in rice plants as did A257. FIG. 7 shows ShB disease severity observations from the 2023 greenhouse trials. The rice plants used in this trial (cultivar Bengal) were grown from seeds treated with buffer (10 mM MgCl₂), with A257, with A257ΔqsmR, or with a commercial seed-treatment fungicide. Plants grown from both A257-treated and A257ΔqsmR-treated seeds showed significantly reduced ShB symptoms as compared either to the buffer control or to a commercial seed-treatment fungicide. Indeed, the commercial fungicide appeared to be essentially ineffective in this trial. Additional trials with the mutant A257ΔqsmR strain will further confirm the efficacy of A257ΔqsmR, to bolster the conclusion that the qsmR-deleted mutant can be used in lieu of A257, with minimal risk of genetic reversion to wild-type virulence.

### 2023 Field Data

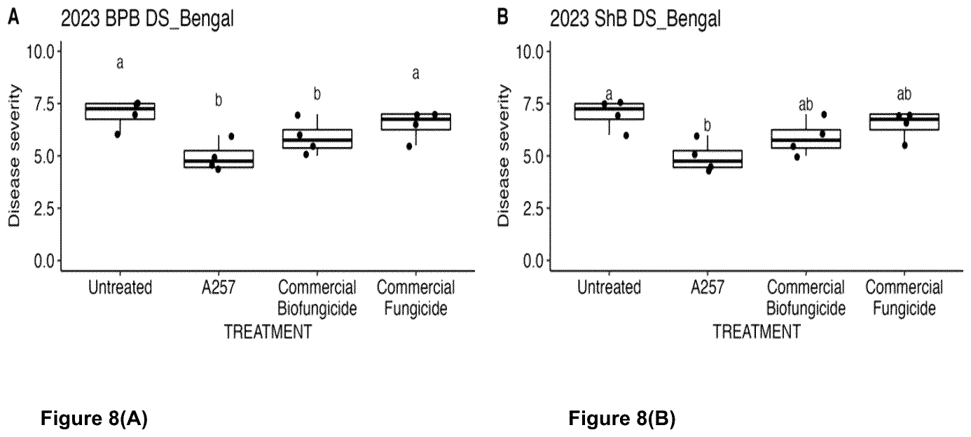

In 2023, the effect of A257 seed treatment was evaluated against both bacterial panicle blight (BPB) and sheath blight (ShB) in field trials at the H. Caffey Rouse Rice Research Station. As of Jul. 20, 2023 (shortly before the priority provisional filing date) significant protective effects had been seen against both diseases. See FIGS. 8(A) and 8(B), which show disease severity observations for various treatments in these trials for BPB and ShB, respectively. Neither commercial seed-treatment (one of which was a biofungicide, and one of which was an organic chemical fungicide) showed significant efficacy against ShB. The commercial biofungicide, however, did show significant efficacy against BPB, at a level comparable to that seen for A257.

### Effect of A257 Seed Treatment on Drought Tolerance

Following a 7-day drought stress, rice plants grown from A257-treated seeds recovered from drought damage, while plants grown from untreated seeds did not. The drought condition was imposed unintentionally due to an accidental leakage of a container that held rice pots and water. This observation suggests that seed treatment with A257 is also effective to increase the tolerance of rice plants to drought, and perhaps other abiotic stresses as well. Similar results are also expected with strains of Burkholderia glumae wherein the qsmR gene has been artificially deleted or otherwise artificially inactivated, for example strain A257ΔqsmR.

### Miscellaneous

The complete disclosures of all references cited herein are hereby incorporated by reference in their entirety. Also incorporated by reference is the complete disclosure of provisional priority application 63/530,287. Also incorporated by reference are the following two works by the inventor and colleagues: J. Bruno et al., Development of alternative materials and strategies for enhancing rice health (Abstract), Plant Health 2022 Conference (Aug. 6-10, 2022); and J. Bruno et al., Development of alternative materials and strategies for enhancing rice health (poster), Plant Health 2022 Conference (Aug. 6-10, 2022) In the event of an otherwise irreconcilable conflict, however, the present specification shall control over material that is incorporated by reference.

The complete genome of strain A257 was described in in Lee, H. H., Lelis, T., Ontoy, J., Bruno, J., Ham, J. H., & Seo, Y. S. (2021). Complete Genome Sequence Data of Four Burkholderia glumae Strains Isolated from Rice Fields in the United States. Mol Plant Microbe Interact, 34 (11), 1324-1327. Furthermore, the complete primers and protocols used to prepare strain A257ΔqsmR from the A257 strain are described in this specification. It is therefore believed that formal deposits of these strains with a depositary should not be required under the patent statute. However, if the USPTO should determine otherwise, then Applicant stands ready to deposit either or both of these strains with a depositary recognized under the Budapest Treaty on the International Recognition of the Deposit of Microorganisms for the Purposes of Patent Procedure. Both strains will be maintained by the Applicant in the interim, and have been maintained by the Applicant since prior to the provisional priority date for this application.

What is claimed:

1. A strain of Burkholderia glumae, wherein said strain is an A257-derived strain in which the qsmR gene has been artificially deleted or otherwise artificially inactivated.

2. The strain of claim 1, wherein said strain is the strain A257ΔqsmR.

3. The strain of claim 1, wherein the qsmR gene has been artificially deleted.

4. The strain of claim 1, wherein the qsmR gene has been artificially inactivated.

5. A method for enhancing the resistance of rice plants to disease or abiotic stress, said method comprising treating rice plants or rice seeds with the strain of claim 1, or with Burkholderia glumae strain A257.

6. The method of claim 5, wherein the strain is the strain A257.

7. The method of claim 5, wherein the strain is the strain A257ΔqsmR.

8. The method of claim 5, wherein said treating step comprises spraying leaves of rice plants.

9. The method of claim 5, wherein said treating step comprises inoculating rice seeds.

10. The method of claim 5, wherein said method enhances the resistance of rice plants against the disease bacterial panicle blight.

11. The method of claim 5, wherein said method enhances the resistance of rice plants against the disease sheath blight.

12. The method of claim 5, wherein said method enhances the resistance of rice plants against the disease narrow brown leaf spot.

13. The method of claim 5, wherein said method enhances the resistance of rice plants against the disease kernel smut.

14. The method of claim 5, wherein said method enhances the resistance of rice plants against the disease false smut.

15. The method of claim 5, wherein said method enhances the resistance of rice plants against the disease blast.

16. The method of claim 5, wherein said method additionally enhances the resistance of rice plants against damage by insects.

17. The method of claim 5, wherein said method enhances the resistance of rice plants to drought.

18. A method for enhancing the resistance of plants to disease or abiotic stress, said method comprising treating plants or seeds with the strain of claim 1, or with Burkholderia glumae strain A257.

19. The method of claim 18, wherein the plants or seeds are soybeans.

20. The method of claim 18, wherein the plants or seeds are selected from the group consisting of maize, wheat, cotton, tomato, and pepper.

21. The method of claim 18, wherein the strain is the strain A257ΔqsmR.

22. The method of claim 18, wherein the strain is the strain A257.

23. A composition of matter comprising rice seeds, maize seeds, wheat seeds, cotton seeds, soybean seeds, tomato seeds, or pepper seeds inoculated with the strain of claim 1.

24. The composition of claim 23, wherein said seeds comprise rice seeds inoculated with the strain of claim 1.

25. The composition of claim 23, wherein said seeds comprise soybean seeds inoculated with the strain of claim 1.


SEQUENCE LISTING (6 sequences)

SEQ ID NO: 1 - DNA, 33 bp, synthetic construct
  source 1..33: mol_type = other DNA
ggatccgggatccgtcgatttcatcgccaattt

SEQ ID NO: 2 - DNA, 35 bp, synthetic construct
  source 1..35: mol_type = other DNA
actagtgggactagtcggtcgctgctttattcagt

SEQ ID NO: 3 - DNA, 33 bp, synthetic construct
  source 1..33: mol_type = other DNA
actagtgggactagtgaatggctgctcgagact

SEQ ID NO: 4 - DNA, 35 bp, synthetic construct
  source 1..35: mol_type = other DNA
tctagagggtctagatcatgttcgatctggctgac

SEQ ID NO: 5 - DNA, 20 bp, synthetic construct
  source 1..20: mol_type = other DNA
cgtgctagaacctgagagac

SEQ ID NO: 6 - DNA, 19 bp, synthetic construct
  source 1..19: mol_type = other DNA
atcgtccagagcactttct